(19) United States
(12) Patent Application Publication
(10) Pub. No.: US 2005/0202001 A1
(43) Pub. Date: Sep. 15, 2005

(54) Enhancement of human epidermal melanogenesis

(76) Inventors: Han-Mo Koo, Kentwood, MI (US); George Vande Woude, Ada, MI (US); Matt Vanbrocklin, Rockford, MI (US); Kathryn Eisenmann, Grand Rapids, MI (US)

Correspondence Address: MCKENNA LONG & ALDRIDGE LLP, 1900 K STREET, NW, WASHINGTON, DC, 20006, US

(21) Appl. No.: 10/512,445

(22) PCT Filed: Apr. 24, 2003
(86) PCT No.: PCT/US03/12825

§ 371 Date: Oct. 25, 2004

Related U.S. Application Data
(60) Provisional application No. 60/374,832, filed on Apr. 24, 2002.

(51) Int. Cl. A61K 38/48; C12N 9/00

(52) U.S. Cl. 424/94.64

(57) ABSTRACT

The invention provides methods and compositions that promote sunless tanning in human skin using a MAPK path-way inhibitor, particularly, a MEK-inhibitor, which induces melanogenesis in epidermal melanocytes alone or in combination with a cAMP-elevating agent. The compositions are preferably administered topically to the skin.

### BACKGROUND OF THE INVENTION

1. Field of the Invention

The invention in the field of biochemistry, medicine and cosmetics relates to methods of increasing pigmentation in human skin by inhibition of the mitogen-activated protein kinase (MAPK) pathway(s) and optionally by elevating the level of intracellular cAMP.

2. Description of the Background Art

Human skin consists of two layers, the uppermost of which is the epidermis. Of the different cell types in the epidermis, the most abundant are keratinocytes. Melanocytes are specialized cells in the basal layer of the epidermis which synthesize melanin; the melanin is then packaged into melanosomes and transported into keratinocytes for storage. Human skin pigmentation occurs as a result of melanin production (melanogenesis) in the melanocytes interspersed throughout the epidermis.

In non-human mammals (e.g., rodents), melanocytes aggregate inside hair follicles and, as such, control only coat (fur) color rather than skin color (Gibbs, P E et al., 1941, Anat Rec 80:61-81). For this reason, humans are the only mammals that tan upon exposure to ultraviolet (UV) light.

Human tanning has evolved as a defense mechanism to protect the skin against the adverse effects of UV exposure which causes inflammation (sunburn) and contributes to aging and malignant melanoma (Galibert, M D et al., 2001, EMBO J. 20(17): 5022-5031). Proteins and nucleic acids of the epidermis absorb UV-B rays. One consequence of UV exposure is the formation of thymine dimers in nuclear DNA which are excised by endonucleases. If not removed, dimers can stall DNA replication generating regions of single-stranded DNA and impairing cell function and growth. Failure to remove thymine dimers may also lead to somatic mutations and increase the risk of carcinogenesis. (Young, A R et al., 1998, J. Invest. Dermatol. 111:982-988).

Furthermore, light-skinned persons are more susceptible than dark-skinned persons to sun-induced skin cancer, face a higher risk of melanoma, and are subject to photoaging or dermatoheliosis, a condition characterized by wrinkling, irregular pigmentation, and surface roughness. However, even darker skinned individuals have a high risk of skin cancer and exacerbated symptoms of aging when subject to prolonged solar UV-B exposure.

Some humans suffer from abnormal skin pigmentation conditions such as vitiligo, piebaldism, albinism, and other hypopigmentation disorders. The most extreme skin pigmentation disorder is total depigmentation of both hair and skin. In less severe instances, regional hypopigmentation results in patchy white areas of skin and hair. These largely cosmetic conditions can cause psychological problems (Arndt, R A et al., eds, Cutaneous Medicine and Surgery, 1996, W.B. Saunders Co: Philadelphia, chapter by Bolognia J L et al, pp. 1219-1232; Torres, A R et al., In Arndt et al., supra, pp. 1233-1241).

Melanin (eumelanin), a polymer of indolequinone, serves as a UV filter by absorbing light within the UV-spectral range (100-400 nm) and thereby provides photoprotection (Glickinan, S L et al., 1997, SPIE Proceedings of Laser-Tissue Interaction VIII, Jacques, J A (ed.) 2975:138-145).

Melanogenesis

The production of melanin (melanogenesis) occurs in organelles that are unique to melanocytes, called melanosomes (Jimbow, K et al., 1998, The Pigmentary System, Norlund, J J et al., eds., Oxford University Press, New York, 1998, pp. 107-114). Three key enzymes involved in melanogenesis are tyrosinase, and the tyrosinase related proteins TRP-1 and TRP-2 (Oetting, W S et al., 1998, The Pigmentary System, supra at pp. 231-249). Of these, tyrosinase is the most critical and is rate-limiting (Hearing, V et al., 1987, Meth. Enzymol 142:154-165; Hearing, V et al., 1987, Int. J. Biochem. 19:1141-1147). Tyrosinase catalyzes the primary step in melanin biosynthesis by hydroxylating tyrosine to dihydroxyphenylalanine (DOPA) and subsequently catalyzing the oxidation of DOPA into dopaquinone. Further transformation of dopaquinone proceeds in two directions: either spontaneous transformation to indolequinone which polymerizes into eumelanin (Olivares C et al., 2001, Biochem J 354:131-9) or transformation into dopachrome which is tautomerized by the dopachrome tautomerase TRP-2, into dihydroxyindole or dihydroxyindole-2-carboxylic acid (DHICA), both of which are converted into indolequinone to form eumelanin (Yokoyarna K et al., 1994, J. Biol. Chem. 289: 27080-27087). TRP-1 influences tyrosinase activity by stabilizing and complexing with this enzyme (Kobayashi K et al., 1998, J. Biol. Chem. 273:31801-31805 and Orlow S J et al., 1994, J. Invest. Dermatol. 103:196-201).

The melanocyte-specific microphthalmia-associated transcription factor (MITF) controls the transcription of tyrosinase, TRP-1 and TRP-2 by binding to the M box sequences of their encoding gene promoters. (Yasumoto, K et al., 1997, J. Biol. Chem. 272: 503-509).

In agouti mice, increases in cyclic AMP (cAMP) levels stimulate MYFF expression and increase melanin production. cAMP activates protein kinase C-β which undergoes nuclear translocation and activates cAMP responsive element (CRE) binding protein (CREB). Activated CREB binds to the CRE promoter of the MITF gene, and drives its transcription, leading to increased tyrosinase expression (B3usca, R et al., 2000, Pigment Cell Res 13:60-69).

Role of Agents that Elevate cAMP

cAMP-elevating agents are known to induce differentiation accompanied by melanin production in murine melanoma cells (Busca et al., supra). Methyl xanthines, for example, isobutylmethylxanthine (IBMX) and other cAMP-elevating agents such as cholera toxin (an adenosine diphosphate ribosyltransferase), α-melanocyte-stimulating hormone, and forskolin (an adenylyl cyclase stimulator), all elevate intracellular cAMP levels in murine melanin producing cells (Englaro, W et al., 1998, J. Biol. Chem. 273:9966-9970).

Sunless Pigmentation of Mammalian Skin

In U.S. Pat. No. 5,643,556, Gilchrest, B et al., disclose a method to induce sunless pigmentation in mammalian epidermis by the topical application of DNA fragments, either single- or double-stranded, or a mixture of both, or deoxynucleotides, dinucleotides, or dinucleotide dimers, in a liposome preparation or in propylene glycol.

In U.S. Pat. Nos. 5,750,091 and 5,352,440 Gilchrest, B et al., disclose a method of inducing sunless pigmentation by topical or subcutaneous (s.c.) administration of diacylglycerol which induces melanogenesis by up-regulating protein kinase C which participates in the cascade that leads to the transcription of tyrosinase.

In U.S. Pat. No. 5,905,091, Fuller, B. et al., disclose a pharmaceutical composition comprising a plurality of prostaglandins and a method of using the pharmaceutical composition to promote sunless tanning.

Alternatively, in U.S. Pat. No. 5,962,417, Gilchrest, B. et al., disclose methods whereby vertebrate skin, hair, wool or fur may be lightened by regulating pigmentation by administration of peptides, antibodies, antibody fragments or DNA sequences encoding peptides that competitively inhibit tyrosinase activation by protein kinase C-β by mimicking protein kinase C-β binding sites on tyrosinase.

MAPK Pathways and MEK

The MAPK superfamily signaling pathways are found in, and highly conserved among, all eukaryotes. These pathways play an integral role in the transduction of various extracellular signals into the nucleus. The best-characterized mammalian pathway, designated Raf-MEK1/2-ERK1/2, also called the Raf/MEK/ERK pathway, includes the MAPK enzymes also known as ERK1 and ERK2, which are phosphorylated and activated by the dual-specificity kinases that have been termed “MAPK/ERK kinases” (abbreviated variously as MAPKK1 and MAPKK2 or; as will be used herein, MEK1 and MEK2). The MEK enzymes are in turn phosphorylated and activated by Raf kinases (Lewis, T S. et al., 1998, Adv. Canc. Res., 74:49-139). The Raf kinase family can be activated by the Ras-GTPase and several other upstream activators, which includes Raf-1, A-Raf and B-Raf. These three kinases are thought to have overlapping, yet unique biochemical functions. Raf-1 and B-Raf can effectively phosphorylate and activate MEK1 and MEK2, whereas A-Raf is a weak activator of MEK1/2. Closely related MEK1 and MEK2 are dual-specificity kinases that phosphorylate and activate ERK1 and ERK2.

Another major set of pathways in the MAPK superfamily pathway involve (1) MEK-3/6 and p38 and (2) MEK-4/7 and JNK/SAPK (JNK is c-Jun N-terminal protein kinase and SAPK is stess-activated protein kinase). For an overview, see: Lee, J T, Jr. and McCubrey, J T: Expert Opin. Ther. Targets, 2002 6(6):659-678 (incorporated herein by reference in its entirety).

The MAPK superfamily pathways are involved in the regulation of cell growth, survival, and differentiation (Lewis et al., supra; Lee & McCubrey, supra). Activated MAPK and/or elevated level of MAPK expression have been detected in a variety of human tumors (Hoshino, R et al., 1999, Oncogene 18:813-822; Salh, B et al., 1999, Anticancer Res. 19:741-48; Sivaraman, V S et al., 1997, J. Clin. Invest. 99:1478-483; Mandell, J W et al., 1998, Am. J. Pathol. 153:1411-23; Licato, L L et al., 1998, Digest. Dis. Sci. 43:1454-1464) and may be associated with invasive, metastatic and angiogenic activities of tumor cells.

With regards to melanogenesis as studied in murine models, cAMP participates in two key pathways: the activation of MAP kinases, which suppress melanogenesis (more below), and the activation of protein kinase C, which induces melanogenesis. This antagonistic relationship that has been observed in murine cells serves as a feedback control that prevents over-expression of melanin which can be toxic in high amounts (Busca et al., supra at 67-68). cAMP-elevating agents stimulate murine melanogenesis independently of MAP kinases. However, MAP kinases have also been implicated in cAMP-induced melanogenesis in B16 murine melanoma (Englaro et al., supra). It is noteworthy that, the present inventors did not observe such an antagonistic relationship between cAMP and MAPK pathway in human melanocytes (or melanoma cells).

MAP kinases promote cellular proliferation which limits melanocyte dendricity and melanogenesis. Conversely, inhibition of the MAP kinase pathway using MEK-inhibitors, such as PD98059, triggers dendrite outgrowth, stimulates the tyrosinase promoter, thus promoting melanogenesis, and enhancing the effects of cAMP on these phenotypes (Englaro et al., supra).

The present inventors and their colleagues found that the Bacillus anthracis lethal factor, LF, a MEK-directed protease (Duesbery, N S et al., 1998, Science 280:734-737; Vitale G et al., 1998, Biochem. Biophys. Res. Comm. 248:706-711), and that PD98059, a small molecule MEK inhibitor, among others, (Dudley D T et al., 1995, Proc. Natl. Acad. Sci. USA 92:7686-7689; Alessi, D R 1995, et al., J. Biol. Chem. 270:27489-27494) stimulated melanogenesis in normal human neonatal melanocytes and human melanoma cells (Koo, H M et al., 2002, Proc. Natl. Acad. Sci. USA 99: 3052-3057).

In addition, while cytotoxic to melanoma cells, MEK-inhibitors are not cytotoxic to normal human melanocytes (Koo, H M et al., supra). In normal melanocytes (as well as keratinocytes), MEK inhibition completely blocks the activation of MAPK, arresting the cells in G₁. However, apoptosis was not detected even after prolonged inhibition (in contrast to the result with human melanoma cells).

Studies showing the effects of MEK-inhibitors and cAMP-elevating agents on melanogenesis have been confined to murine models and to murine coat color (see Englaro, supra; Blanchard, S G, 1995, Biochemistry 34:10406-10411).

There is a need in the art for new remedies for hypopigmentation and for general cosmetic enhancement, such as a tanned appearance, without exposure to the damaging effects of UV irradiation or the toxic side effects of a number of “tanning formulas” that have been available (Bluhm, R et al., 1990, JAMA 264(9): 1141-1142; and Goodheart, H P 1999, Woman's Health in Primary Care 2: 865-866).

Moreover, it has not been suggested, prior to the present invention, that MEK-inhibitors, cAMP-elevating agents, or a combination of the two, could be used in human skin to stimulate melanogenesis and, thereby, to mimic the effects of sun tanning.

### SUMMARY OF THE INVENTION

The present inventors have discovered that the administration of a MEK-inhibitor and, optionally, a cAMP-elevating agent to human skin effectively induces melanogenesis and pigmentation therein and is therefore useful in a method of sunless tanning.

Although the exemplification herein focuses on melanogenesis stimulated by MEK inhibitors, the present inventors have conceived that targeting other kinases of the MAPK superfamily pathway, particularly upstream Raf and downstream ERK1/2 with inhibitors produces the same types of melanogenic effects. Thus, inhibitors of these other enzymes in the pathway are also useful for sunless tanning. Inhibitors may be targeted to MAPK pathway enzyme expression, enzymatic activity, or both, and may act directly or indirectly, for example, by selective inhibition of transcription or translation of the enzyme's mRNA or protein, inhibition of post-translational modification (e.g., farnesylation or geranyl-geranylation), inhibition of intracellular trafficking or processing of the enzyme, competitive or noncompetitive inhibition of the enzyme's catalytic activity, etc. Thus, the classes of inhibitors may be manifold, including those described in Lee and McCubrey, supra, all of which MAPK pathway inhibitors are intended to be within the scope of this invention. Thus, small organic molecules, selective proteases, monoclonal antibodies, aptamers, antisense oligonucleotides, siRNAs, and the like, as long as they inhibit the expression or activity of a MAPK pathway enzyme, are useful as inducers or promoters or epidermal melanocyte melanogenesis, and therefore, of sunless tanning. These include known molecules and molecules yet to be discovered. Their utility in accordance with the present invention can be tested readily using the experimental methods disclosed and exemplified herein.

The present invention provides a method of promoting sunless tanning by inducing melanogenesis in human skin.

In this method, a MAPK superfamily pathway inhibitor such as a Raf inhibitor, a MEK-inhibitor or and ERK1/2 inhibitor, or a combination thereof, is or are administered to the human epidermis, thereby inhibiting the MAPK pathway and, inter alia, stimulating transcription of melanogenic enzymes, which promotes melanogenesis and subsequent enhancement in the level of visible pigmentation.

In another embodiment the selected MAPK pathway inhibitor is co-administered with a cAMP-elevating agent, which increases the level of intracellular cAMP, and synergistically enhances melanogenesis. Preferred MAPK pathway inhibitors are MEK inhibitors.

The preferred small molecule MEK-inhibitor is PD98059.

In other embodiments the MEK-inhibitors are U0126 and PD184352.

In yet another embodiment the MEK-inhibitor is a protein or polypeptide, a preferred example of which is Bacillus anthracis lethal factor (LF), administered as B. anthracis lethal toxin (LeTx), which is in combination with the B. anthracis protein known as “protective antigen” that permits insertion of the LF into the cell.

Preferred cAMP elevating agents are adenylyl cyclases, agents that increase cyclase activity, or phosphodiesterase inhibitors. B. anthracis edema toxin (an adenylyl cyclase) is one disclosed cAMP elevating agent. Other preferred cAMP-elevating agents include isobutylmethylxanthine (a phosphodiesterase inhibitor), forskolin (an adenylyl cyclase activator) and cholera toxin (an adenosine diphosphate ribosyltransferase).

The present objectives can be achieved by topical (or s.c.) application of various MEK-inhibitory melanogenic compounds, alone or in combination with cAMP elevating agents. For this purpose, the invention also provides a topical pharmaceutic/cosmetic composition comprising a MEK-inhibitor alone or in combination with a cAMP elevating agent.

### BRIEF DESCRIPTION OF THE DRAWINGS

FIG. 1 shows comparative melanin production in human epidermal melanocytes as determined by visual inspection of a lysed cell solution in a microcentrifuge tube after treatment with the MEK-inhibitor PD98059, versus a DMSO solvent control.

FIGS. 2A and 2B are graphs showing the comparative levels of melanin production in melanoma cells treated with MEK-inhibitors, cAMP-elevating agents; or both, versus controls. FIG. 2A: UACC-257 melanoma cells; and FIG. 2B: MALME-3M melanoma cells; both groups were treated with (1) B. anthracis protective antigen (PA) alone as a control, (2) B. anthracis lethal toxin (LeTx; which is lethal factor (LF) together with PA), (3) B. anthracis edema toxin (EdTx; which is B. anthracis edema factor together with PA), and (4) LeTx and EdTx together. Also tested were (5) DMSO solvent as a control, (6) PD98059, (7) isobutylmethylxanthine (IBMX), or (8) PD98059+IBMX. The melanin content (A₄₀₅/A₂₈₀) is expressed as an arbitrary ratio unit compared to the PA control, which is set at 1.0. The upper panel photographs depict the amount of pigmentation of corresponding UACC-257 cells in the wells of a 96-well plate.

FIGS. 3A and 3B show melanin production in human melanoma tissue treated with B. anthracis LeTx. FIG. 3A shows SK-MEL-28 human melanoma cells grown in athymic nude mice and treated with B. anthracis LeTx by s.c. injection. A tissue section stained with hematoxylin and eosin is shown with melanin deposits at higher magnification (inset). FIG. 3B shows M14-MEL human melanoma cells grown in athymic nude mice which were injected s.c. with B. anthracis LeTx. The visible dorsal dark melanin patch under the skin shows where melanoma cells were growing.

### DESCRIPTION OF THE PREFERRED EMBODIMENTS

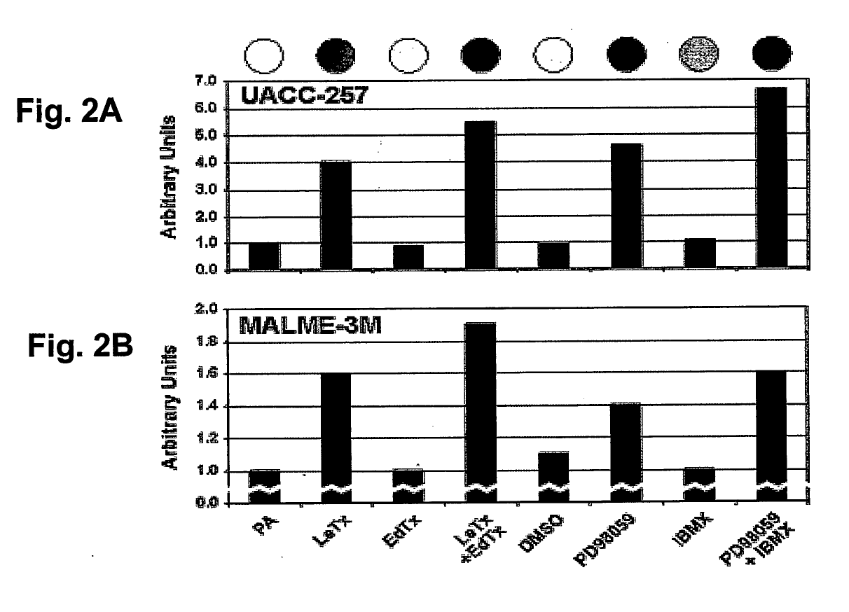

The inventors discovered that, in contrast to previously reported results in mice, human epidermal melanocytes treated with cAMP-elevating agents alone produced little or no melanin or visible pigmentation. They concluded that cAMP elevating agents alone are not useful as stimulators of sunless tanning in humans. The present invention is based on the conception that exposure to sunlight (UV irradiation) may stimulate melanin production in human skin melanocytes by inhibiting activity of the MAPK pathways together with elevating cAMP levels, consequently leading to a tanned appearance in humans. The key discovery described herein is that MEK inhibitors stimulate melanogenesis in normal human melanocytes and that the addition of cAMP-elevating agents, exemplified as B. anthracis edema factor (EF) or IBMX, increase melanogenesis by about 20% (see FIG. 2A/2B). Thus, sunless tanning can be achieved by the use of a MAPK pathway inhibitor, preferably a MEK inhibitor, and more preferably, the combination of a MEK inhibitor and a cAMP-elevating agent. Such a combination will increase the pigmentation of human skin by increasing the melanin content of melanocytes residing in the skin while avoiding the many undesirable effects of UV exposure.

Inhibitors of MEK

I. MEK-Directed Proteases

The term “MEK-directed protease activity” refers to the proteolytic activity of a protease on MEK1 resulting in the inactivation of MEK1. This term is intended to include protease activity on any member of the MEK family. The designation MEK refers to a family of protein kinases that are part of the MAPK pathway. Examples are MEK1, MEK2 and MEK3, etc. These proteins share sequence similarity, particularly at the N-terminus. See, for example, Duesbery N S et al., 1999, CMLS Cell. Mol. Life Sci. 55:1599-1609.

Thus, a “MEK-directed protease” or “MEK protease” refers to
(1) a protease acting on members of the MEK protein family,
(2) a protease that acts on conservative amino acid substitution variants or other conservatively modified variants thereof; and
(3) a protease that acts on allelic or polymorphic MEK variants, muteins and homologues in other species with greater than about 60%, preferably greater than about 70%, more preferably greater than about 80% and most preferably greater than about 90% sequence identity to MEK1, MEK2, MEK3, etc.

In one embodiment, MEK (i.e., MEK1 and MENK2) is inhibited by B. anthracis lethal toxin (LeTx), which comprises B. anthracis lethal factor and protective antigen.

While the present disclosure focuses on the use of B. anthracis LF as a MEK pathway inhibitor, it is to be understood that homologues of LF from other Bacillus species and mutants thereof that possess the characteristics disclosed herein are intended within the scope of this invention.

Also included is a “functional derivative” of LF, which means an amino acid substitution variant, a “fragment,” or a “chemical derivative” of LF, which terms are defined below. A functional derivative retains at least a portion of the relevant LF activity, that of proteolysis of MEK1 which permits its utility in accordance with the present invention.

A “variant” of the MEK-directed protease refers to a molecule substantially identical to either the full protein or to a fragment thereof in which one or more amino acid residues have been replaced (substitution variant) or which has one or several residues deleted (deletion variant) or added (addition variant). A “fragment” of the MEK-directed protease refers to any subset of the molecule, that is, a shorter polypeptide of the fall length protein.

A preferred group of MEK-directed protease variants are those in which at least one amino acid residue and preferably, only one, has been substituted by different residue. For a detailed description of protein chemistry and structure, see Schulz, G E et al., Principles of Protein Structure, Springer-Verlag, N.Y., 1978, and Creighton, T E Proteins: Structure and Molecular Properties, W.H. Freeman & Co., San Francisco, 1983, which are hereby incorporated by reference. The types of substitutions that may be made in the protein molecule may be based on analysis of the frequencies of amino acid changes between a homologous protein of different species. Based on such an analysis, conservative substitutions are defined herein as exchanges within one of the following five groups:

| | | |
|---|---|---|
| 1 | Small aliphatic, nonpolar or slightly polar | Ala, Ser, Thr (Pro, Gly); |
| | residues | |
| 2 | Polar, negatively charged residues and | Asp, Asn, Glu, Gln; |
| | their amides | |
| 3 | Polar, positively charged residues | His, Arg, Lys; |
| 4 | Large aliphatic, nonpolar residues | Met, Leu, Ile, Val (Cys); |
| 5 | Large aromatic residues | Phe, Tyr, Trp. |

The three amino acid residues in parentheses above have special roles in protein architecture. Gly, the only residue lacking a side chain, imparts flexibility to the chain. Pro, because of its unusual geometry, tightly constrains the chain. Cys, can participate in disulfide bond formation which is important in protein folding.

More substantial changes in biochemical or other functional properties are made by selecting substitutions that are less conservative, such as between; rather than within, the above five groups. Such changes will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ele, Phe, Val or Ala; (iii) substitution of a Cys residue for (or by) any other residue; (iv) substitution of a residue having an electropositive side chain, e.g., Lys, Arg or His, for (or by) a residue having an electronegative charge, e.g., Glu or Asp; or (v) substitution of a residue having a bulky side chain, e.g., Phe, for (or by) a residue not having such a side chain (e.g., Gly).

Most acceptable deletions, insertions, and substitutions according to the present invention are those that do not produce radical changes in the characteristics of the protein in terms of its proteolytic activity or its MEK-inhibitory and/or melanogenic activity. However, when it is difficult to predict the exact effect of the substitution, deletion, or insertion in advance of doing so, one skilled in the art will appreciate that the effect can be evaluated by routine screening assays such as those described here, without requiring undue experimentation.

Whereas shorter chain variants can be made by chemical synthesis, for the present invention, the preferred longer chain variants are typically made by site-specific mutagenesis of the nucleic acid encoding the polypeptide, expression of the variant nucleic acid in cell culture, and, optionally, purification of the polypeptide from the cell culture, for example, by immunoaffinity chromatography using a specific antibody immobilized to a column (to absorb the variant by binding to at least one epitope).

The activity of a variant present in a cell lysate or a more highly purified preparation is screened in a suitable screening assay for the desired characteristic, preferably the proteolysis of MEK1 and, ultimately, the stimulation of melanogenesis in human melanocytes. It is also possible to follow the immunological character of the protein molecule assayed by alterations in binding to a given antibody, and may be measured by competitive immunoassay. Biochemical or biological activity is screened in an appropriate assay, as described below.

A “mimetic” of a MEK-directed protease is an agent, generally a polypeptide or peptide molecule, that recognizes MEK, e.g., MEK1, as a substrate and cleaves MEK1 at the same site cleaved by full-length, native protease such as LF. Thus, such mimetics include homologues, peptides, conservative substitution variants, as well as deletion variants that retain the protease active site and proteolytic action on MEK1. Such mimetics are tested using assays for protease activity, e.g., MEK1 mobility shift assays, MOS-induced activation of MAPK in oocytes and myelin basic protein (NMP) phosphorylation, as described below in a melanogenesis assay, preferably using normal human melanocytes. In assessing a mimetic, LF is generally the positive control for protease activity or melanogenic activity. A mimetic has at least about 25% of the activity of this positive control, more preferably at least about 50-100% of the activity. Of course, the mimetic can also have >100% of the activity of this control.

Also useful in the present methods are agents that potentiate or promote the above proteolytic activity that, together with LF, LF homologues or mimetics, promote their melanogenic activity. A “potentiator” of the protease is an agent that activates (promotes, enhances, or increases) the proteolytic activity and is identified by in vitro or in vivo assays of this activity or downstream activities in the MAPK pathway.

Samples that are treated with a candidate protease potentiator are compared to control samples that have not been treated with the test compound. This permits assessment of the presence and extent of activation of MEK1 protease activity. Control samples (untreated with test compounds) are assigned a relative protease activity value of 1. Activation is achieved when the measured protease activity value is about 1.5, more preferably 2.0 or greater. Potentiatiors can also be evaluated in a cellular assay of MEK action, for example an assay for melanogenesis in human melanocytes or growth inhibition or apoptosis of human melanoma cells in culture.

Chemical Modification of the Protein

A “chemical derivative” of a MEK-directed protease contains additional chemical moieties not normally part of the protein. Covalent modifications of the protein are included within the scope of this invention. Such modifications may be introduced into the molecule by reacting targeted amino acid residues with an organic derivatizing agent capable of reacting with selected side chains or terminal residues. Such chemically modified and derivatized moieties may improve the protein's solubility, absorption, biological half life, and the like. These changes may eliminate or attenuate undesirable side effects of the protein in vivo. Moieties capable of mediating such effects are disclosed, for example, in Remington 's Pharmaceutical Sciences, Mack Publishing Company, Easton Pa. (Gennaro, 18th ed. 1990).

Preparation of Recombinant MEK-directed protease (and MEK1) Proteins

As described herein, native or recombinant MEK-directed protease proteins, their homologues and mimetics are used in the methods of the invention. MEK1, the target of proteolytic activity, may also be provided in native or recombinant form for testing. Recombinant proteins may be particularly convenient for biochemical assays. MEK-directed protease homologues and functional derivatives such as substitution variants and fusion proteins may be prepared recombinantly for evaluation of their mimetic activity as stimulators of melanogenesis. Recombinant proteins are prepared by conventional means which are generally described below along with methods for biochemical isolation and purification of the proteins from natural sources.

General Recombinant DNA Methods

Basic texts disclosing general methods of molecular biology, all of which are incorporated herein by reference, include: Sambrook, J et al., Molecular Cloning: A Laboratory Manual, 2nd Edition, Cold Spring Harbor Press, Cold Spring Harbor, N.Y., 1989; Ausubel, F M et al., Current Protocols in Molecular Biology, Vol. 2, Wiley-Interscience, New York, (current edition); Kriegler, Gene Transfer and Expression: A Laboratory Manual (1990); Glover, D M, ed, DNA Cloning: A Practical Approach, vol. I & II, IRL Press, 1985; Albers, B. et al., Molecular Biology of the Cell, 2nd Ed., Garland Publishing, Inc., New York, N.Y. (1989); Watson, J D et al., Recombinant DNA, 2^{nd} Ed., Scientific American Books, New York, 1992; and Old, R W et al, Principles of Gene Manipulation: An Introduction to Genetic Engineering, 2^{nd} Ed., University of California Press, Berkeley, Calif. (1981).

Unless otherwise indicated, a particular nucleic acid sequence additionally encompasses conservative substitution variants thereof (e.g., degenerate codon substitutions) and a complementary sequence. The term “nucleic acid” is intended to include a gene, cDNA, mRNA, an oligonucleotide (any polynucleotide). Sizes of nucleic acids are stated either as kilobases (kb) or base pairs (bp). These are estimates derived from agarose electrophoresis or polyacrylamide gel electrophoresis (PAGE), from sequences of nucleic acids which are determined by the user or published. Protein sizes are stated as molecular mass in kilodaltons (kDa) or as length (number of amino acid residues). Proteins sizes are estimated from PAGE, from sequencing, from presumptive amino acid sequences based on nucleic acid sequence, or from published amino acid sequences.

Oligonucleotides that are not commercially available may be chemically synthesized (Oligonucleotide Synthesis, Gait N, ed., Current Edition), for example, according to the solid phase phosphoramidite triester method (Beaucage S L et al., 1981, Tetrahed. Lett. 22:1859-1862) using an automated synthesizer (Van Devanter N, et. al., 1984, Nucl Acids Res. 12:6159-6168).

Oligonucleotides are purified by native acrylamide gel electrophoresis or by anion-exchange HPLC (Pearson et al., 1983, J. Chromatog. 255:137-149). The sequence of a cloned gene or a synthetic oligonucleotide can be verified using the chain termination method for sequencing double-stranded templates (Wallace et al., 1981, Gene 16:21-26).

Cloning of Nucleic Acids Encoding MEBK-1 Proteases and Other Proteins

In general, a nucleic acid encoding a MEK1 protease, PA, or a homologous nucleic acid is cloned starting from cDNA or genomic DNA libraries or is isolated by polymerase chain reaction (PCR) amplification techniques using oligonucleotide primers. For example, LF is isolated from B. anthracis DNA (genomic or cDNA) libraries. Genes for MEK1 can be cloned from mammalian libraries, preferably human libraries. For example, MEK1 sequences can be isolated from sarcoma libraries from cells with an activated MAPK pathway. PA can be cloned from a B. anthracis DNA library.

Amplification techniques using primers may also be employed to amplify and isolate PA, and MEK1 from DNA or RNA (U.S. Pat. Nos. 4,683,195 and 4,683,202; PCR Protocols: A Guide to Methods and Applications (Innis et al., eds, 1990)). PCR and ligase chain reaction (LCR) methods can be used to amplify a nucleic acid sequences directly from mRNA, cDNA or genomic DNA. Degenerate oligonucleotides are routinely designed to amplify homologues.

II. Small Molecule Inhibitors of MEK

Also intended within the scope of this invention are small organic molecules that act as inhibitors of MEK and stimulators of melanogenesis. As used herein, “small molecules” are organic chemical entities that are not biological macromolecules such as proteins or peptides. The small molecule inhibitors of MEK generally have a molecular mass of less than about 2000 Da, preferably less than about 1000 Da, more preferably less than about 500 Da.

In a preferred embodiment, inhibition of MEK by the small molecule inhibitor PD98059 (also referred to as PD098059) results in the efficient induction of melanogenesis in human epidermal melanocytes.

Other small molecule inhibitors of the MAPK pathway are expected to have the same melanogenic effects on human epidermis as those exemplified herein. These include the MEK inhibitors PD184352 (Sebolt-Leopold, J S et al., Nature Med. 5: 810-816, 1999) and U0126 (Favata, M et al., J Biol. Chem. 273:18623-18632, 1998) and the like. The chemical structures of these compounds are depicted below:

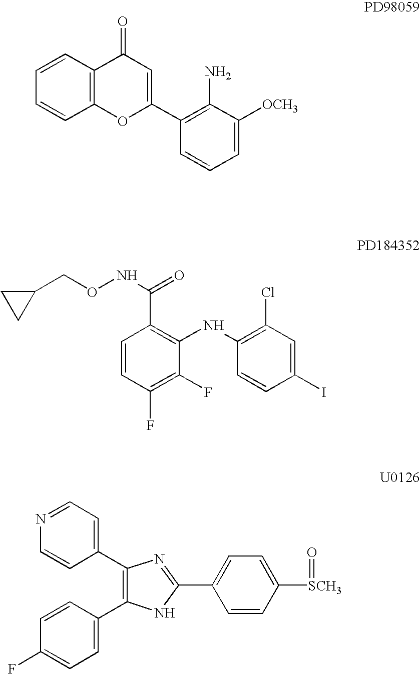

Inhibitors of other enzymes of the MAPK pathway are known, for example Raf and ERK1/2 inhibitors and are similarly used herein. Some of these types of inhibitors are listed and their chemical formulae shown in Lee and McCubrey, supra. Examples include the Raf kinase inhibitors BAY-43-9006, GW-5074 and L-779450. MEK inhibitors in addition to those shown above, include L-783277, Ro-092210, and the “Wyeth-Ayerst inhibitor.”

A number of Raf inhibitors are protein destabilizers, specifically Geldanamycin, KF-25706, KF-58333 and Novobiocin (see Lee and McCubrey, supra, and references cited therein). Various Ras inhibitors are also known, that would impact the pathway upstream of Raf; examples are farnesyltransferase inhibitors (R-11577, SCH-66366, L-778123, BMS-214662, and arglabin-DMA. Antisense oligonucleotide Ras inhibitors include ISIS-5132 and ISIS-3521.

The present invention further includes analogues, derivatives, and cosmetically acceptable salts of the above small molecule MAPK pathway inhibitors, preferably MEK-inhibitors, that have melanogenic affects on human epidermal melanocytes. Derivatives or analogues can be generated by, for example, derivatizing available nitrogens to form amides, ureas, carbamates, or higher amines (e.g., primary to secondary, to tertiary, or to quaternary, etc.). In other analogues and derivatives, aromatic halogen atoms are replaced with, for example, other halogens, mono-, di-, or trihalomethyl, or halomethoxy groups, or alkoxy groups such as methoxy, while maintaining the desired MEK-inhibiting/melanogenesis stimulating activity. Such derivatives or analogues may hydrolyze or metabolize to form the above listed compounds or to form further analogues. Also intended are derivatives and analogues that promote absorption into the skin or assist in transport to an active site. Preferred substitutions are those which promote skin absorption leading to improved activity profiles. Such analogues and derivatives can be prepared through routine experimentation. Similarly, testing for advantageous properties, (e.g., fat solubility or transport into the skin) is routine and within the ordinary skill in the art and is additionally provided by the experimental procedures disclosed herein.

Inhibitors of ERK1 and ERK2

MEK directed phosphorylation and activation of the ERK1/2 enzymes is also responsible for down-regulating the transcription of the melanogenic enzymes tyrosinase, TRP-1 and TRP-2. As such, the present invention includes methods of inducing melanogenesis using an agent that acts as a direct inhibitor (rather than an upstream) inhibitor of ERK1/2. Such an inhibitor that inhibits ERK1/2 enzymatic activity (or otherwise interferes with ERK1/2 synthesis, localization, stability, etc.) whether presently known or later discovered, may be administered alone or in combination with a cAMP-elevating agent. One type of ERK1/2 inhibitor is an ERK phosphatase also known as CL-100 (Tyrrell, R M, 1996, EXS 77:255-271), a human stress-response gene that regulates MAP kinase activity and which has been reported to be upregulated by exposure to cytotoxic levels of UVA radiation. Use of such an inhibitor in accordance with this invention would require introduction of the protein into cells (Fukuda M et al., 1995, Oncogene 11:239-244) or delivery of encoding DNA and its expression within the appropriate cells.

Pharmaceutical/Cosmetic Compositions, Their Formulation and Use

A pharmaceutical composition (which term is intended to include a cosmetic composition) according to this invention comprises a MAPK pathway inhibitor, preferably a the MEK-directed protease (or functional derivative or mimetic) or a small molecule MEK-inhibitor, or an analogue or derivative thereof, in a formulation with a pharmaceutically or cosmetically acceptable carrier or excipient.

Pharmaceutical compositions within the scope of this invention include all compositions wherein the inhibitor, whether a MEK-directed protease, or another small molecule inhibitor, and a cAMP-elevating agent, is or are present in an amount effective to achieve its intended purpose. It may be desirable to combine a MAPK pathway, preferably MEK,-inhibitor and a cAMP-elevating agent into a single formulation, wherein each is at a concentration such that the combination provides an effective dose to stimulate melanogenesis. While individual needs vary, determination of optimal ranges of effective amounts of each component is within the skill of the art. Typical dosages comprise 0.1 to 100 mg/kg/body weight, though more preferred dosages are described for certain particular uses, below.

In addition to the pharmacologically active protein or small molecule (or nucleic acid-based agent), the pharmaceutical compositions may contain suitable pharmaceutically acceptable carriers comprising excipients and auxiliaries which facilitate processing of the active compounds into preparations which can be used topically or for injection; these carriers are well known in the art. Suitable solutions for administration by topically or by subcutaneous injection may contain from about 0.01 to 99 percent, active compound(s) together with the excipient.

The pharmaceutical preparations of the present invention are manufactured in a manner which is known, for example, by means of conventional mixing, granulating, dissolving, or lyophilizing processes. Suitable excipients may include fillers, binders, disintegrating agents, auxiliaries, and stabilizers, all of which are known in the art. In addition, suspensions of the active compounds as appropriate for oily injection suspensions may be administered. Suitable lipophilic solvents or vehicles include fatty oils.

Exemplary oils which may be used include mineral oils (liquid petrolatum), plant oils (liquid fraction of karite butter, sunflower oil, sesame oil), animal oils (perhydrosqualen(e), synthetic oils (purcellin oil), silicone oils (cyclomethicone) and fluoro-oils (perfluoropolyethers). Fatty alcohols, fatty acids (stearic acid) or synthetic fatty acid esters (ethyl oleate or triglycerides) and waxes (paraffin wax, carnauba wax and beeswax) may also be used as fats.

Other possible cosmetically acceptable carriers can include liquid petrolatum, isopropyl palmitate, polyethylene glycol, ethanol (95%), polyoxyethylene monolauriate (5%) in water, sodium lauryl sulfate (5%) in water. Other materials such as anti-oxidants, humectants, viscosity stabilizers (for aqueous injection suspensions), and similar agents may be added as desired. In addition, fragrances may be added to the compositions to improve their scent or colored agents to enhance the appearance of the composition.

Cosmetically Acceptable Carriers and Vehicles

As used herein, a “cosmetically acceptable topical carrier” or a “cosmetically acceptable vehicle” refers to a carrier, a dilutant, or a dispersant capable of delivering a MAPK pathway inhibitor, preferably MEK-inhibitor, and, optionally, a cAMP-elevating agent, to the skin (or to an appropriate layer thereof) without undue toxicity, irritation, allergenicity or the like. In addition, to be cosmetically acceptable, a topical carrier preferably possesses favorable cosmetic properties such as overall feel, ability to be rubbed in to the skin, lack of excessive greasiness, etc.). Most preferred topical carriers are organic materials in which the inhibitor and optional cAMP-elevating agent can be dispersed or dissolved Sagarin, E et al, 1972, Cosmetics, Science and Technology, 2d ed., 1:48-65), incorporated herein by reference, contains numerous examples of suitable cosmetically acceptable topical carriers. Examples include various emollients, emulsifiers, humectants, thickeners and powders, and solvents (including water) as described below.

Examples of cosmetically acceptable organic solvents are propylene glycol, polyethylene glycol (200-600), polypropylene glycol (425-2025), glycerol, sorbitol esters, 1,2,6-hexanetriol, ethanol, isopropanol, butanediol, and mixtures thereof.

The cosmetically acceptable vehicle will usually form from 5% to 99.9%, preferably from 25% to 80% by weight of the active composition, and can, in the absence of other cosmetic adjuncts, form the balance of the composition.

The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase or vise versa; or suspensions or emulsions of smooth, semi-solid or solid consistency of the cream or gel type. These compositions are formulated according to the usual techniques well known in the art.

When a composition of the invention is formulated as an emulsion, the proportion of the fatty phase may range from 5% to 80% by weight, preferably from 5% to 50%, relative to the total weight of the composition. Oils, emulsifiers and co-emulsifiers incorporated in the composition in emulsion form are selected from among those used conventionally in the cosmetic or dermatology field. An emulsifier and coemulsifier may be present in the composition at a proportion ranging from 0.3% to 30% by weight, preferably from 0.5% to 20%, relative to the total weight of the composition.

When the compositions of the invention are formulated as an oily solution or gel, the fatty phase may constitute more than 90% of the total weight of the composition.

The compositions of the invention may also contain additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, fillers, bactericides, odor absorbers and dyestuffs or colorants. The amounts of these various additives and adjuvants are those conventionally used in the art, and, for example, range from 0.01% to 10% of the total weight of the composition. Depending on their nature, these additives and adjuvants may be introduced into the fatty phase, into the aqueous phase.

Hydrophilic gelling agents include carboxyvinyl polymers (carbomer), acrylic copolymers such as acrylate-alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

An oil or oily material may be present, together with an emollient to provide either a water-in-oil emulsion or an oil-in-water emulsion, depending largely on the average hydrophilic-lipophilic balance (BLB) of the emollient employed. Levels of such emollients may range from about 0.5% to about 50%, preferably between about 5% and 30% by weight of the total composition. Emollients may be classified as esters, fatty acids and alcohols, polyols and hydrocarbons.

Esters may be mono- or di-esters. Acceptable fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, di-isopropyl adipate and cetyl octanoate.

Suitable fatty alcohols and acids include those compounds having chains of 10 to 20 carbon atoms. Especially preferred are such compounds such as cetyl, myristyl, palmitic and stearyl alcohols and acids.

Among polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbon has hydrocarbon chains anywhere from 12 to 30 carbon atoms. Specific examples include mineral oil, petroleum jelly, squalene and isoparaffins.

Another category of functional ingredients within the cosmetic compositions of the present invention are thickeners. A thickener will usually be present in amounts anywhere from 0.1 to 20% by weight, preferably from about 0.5% to 10% by weight of the composition. Exemplary thickeners are cross-linked polyacrylate materials available under the trademark Carbopol®. Gums may be employed such as xanthan, carrageenan, gelatin, karaya, pectin, and locust beans gum. Under certain circumstances the thickening function may be accomplished by a material also serving as a silicone or emollient. For instance, silicone gums in excess of 10 centistokes and esters such as glycerol stearate have dual functionality.

Powders may be incorporated into the cosmetic composition of the invention. These powder may include chalk, talc, kaolin, starch, smectite clays, chemically modified magnesium aluminum silicate, organically modified montmorillonite clay, hydrated aluminum silicate, fumed silica, aluminum starch octenyl succinate, and mixtures thereof.

The compositions may be in the form of a lyophilized particulate material, a sterile or aseptically produced solution Vehicles, such as water (preferably buffered to a physiologically acceptable pH, as for example, in phosphate buffered saline) or other inert solid or liquid material such as normal saline or various buffers may be present. The particular vehicle should be selected optimize the composition for topical or subcutaneous administration.

In general terms, a pharmaceutical/cosmetic composition is prepared by mixing, dissolving, binding or otherwise combining the MAPK pathway inhibitor or cAMP elevating agent or both with one or more water-insoluble or water-soluble aqueous or non-aqueous vehicles. If necessary, another suitable additive or adjuvant can be included. It is imperative that the vehicle, carrier or excipient, as well as the conditions for formulating the composition are such that do not adversely affect the biological or pharmaceutical activity of the proteins, peptides or small molecules.

Subjects, Treatment Modalities and Routes of Administration

Humans are the preferred subjects of the present invention. The term “treating” is intended to include administering to subjects a pharmaceutical composition comprising a MEK-inhibitor (whether a protease or a small molecule inhibitor), a carrier suitable for topical administration or injection via another route, e.g., transdermal and; optionally, a cAMP elevating agent in the same or similar carrier. Treating includes administering the pharmaceutical composition to a subject who wishes to increase pigmentation in their skin.

Due to the melanogenic effects of the present methods, this invention is useful for inducing or promoting melanogenesis which results in enhanced pigmentation of the skin for cosmetic purposes, as tanning inducer and accelerator in the presence or absence of natural sunlight, for pigmenting (coloring) skin grafts, allografts, and autografts in vitro and in vivo; for treating hypopigmentation disorders such as vitiligo, albinism, piebaldism, and post-inflammatory hypopigmentation; as a treatment for darkening, or repigmenting human hair, for preventing gray (depigmented) in human hair.

The compositions of the present invention wherein the MEK-directed protease or inhibitor and optionally the cAMP-elevating agent is combined with a pharmaceutically or cosmetically acceptable excipient or carrier, may be administered by any means that achieve their intended purpose. Amounts and regimens for administration can be determined readily by those with ordinary skill in the clinical art of treating any of the above mentioned diseases. Preferred amounts are described below.

In general, the present methods include administration by parenteral routes. Although the most preferred route is topical, the invention includes subcutaneous, intravenous, intramuscular, and intradermal injection or infusion, and transdermal delivery. The dosage administered will be dependent upon the age, health, and weight of the recipient, kind of concurrent treatment, if any, frequency of treatment, and the nature of the effect desired.

In the present method, the compositions can be given once but generally is administered six to twelve times (or even more, as is within the skill of the art to determine empirically without undue experimentation). The treatments can be performed daily (or more than once per day) but are generally carried out every two to three days or as infrequently as once a week, as is beneficial, desired or necessary. Dosage and duration requirements between subjects may vary due to body type, initial level of pigmentation, or desired final pigmentation. Typically between about 1.0 ng—about 1.0 g, preferably about 1.0 μg—about 100 mg, and most preferably between about 100 μg to about 10 mg, of MEK-inhibitor with or without a cAMP-elevating agent of about equal proportion, is included within the composition. In any event, it-would be within routine skill in the art to determine empirically the frequency and/or dosage required to achieve the desired level of enhanced pigmentation.

The inhibitor and cAMP elevating agent is preferably incorporated into topically applied vehicles such as solutions, suspensions, emulsions, oils, creams, ointments, powders, liniments, salves, and the like, as a means for administering the active ingredient(s) directly to the desired area. The carrier for the active agent may be either in sprayable or non-sprayable form. Non-sprayable forms can be semi-solid or solid forms comprising a carrier indigenous to topical application and having a dynamic viscosity preferably greater than that of water. If desired, these may be sterilized or mixed with auxiliary agents, e.g., preservatives, stabilizers, wetting agents, buffers, or salts for influencing osmotic pressure and the like. Examples of preferred vehicles for non-sprayable topical preparations include ointment bases, e.g., polyethylene glycol-1000 (PEG-1000); conventional creams such as HEB cream; gels; as well as petroleum jelly and the like.

For the preferred topical application to human skin, it is preferred to administer an effective amount of a compound according to the present invention to the desired skin surface. This amount will generally range from about 0.001 mg to about 1 g per application, depending upon the nature of the agent, area to be treated, the amount of pigmentation desired, and the nature of the topical vehicle employed. A preferred topical preparation is an ointment wherein about 0.01 mg to about 50 mg of active ingredient is used per ml of ointment base, such as PEG-1000.

Other pharmaceutically acceptable carriers, especially for topical application, are liposomes, pharmaceutical compositions in which the active compounds are either dispersed or variously present in corpuscles consisting of aqueous concentric layers adherent to lipidic layers. The active compounds preferably present in the aqueous layer and in the lipidic layer, inside or outside, or; in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature.

Transdermal and Intradermal Administration

The compounds may also be formulated for transdermal or intradermal administration, for example, in the form of transdermal patches, e.g., for the treatment of vitiligo or other hypopigmentation disorders. Any suitable patch which serves to appropriately deliver the compositions of the invention may be used. The chief requirement is that the patch adheres to the portion of the skin being treated, and that it delivers the MEK-inhibitors and optional cAMP-elevating agents and supporting chemical constituents to the skin. Delayed release mechanisms may be employed to assure long term delivery.

The transdermal patch, well-known in the art, is preferably adhered to the skin using a pressure sensitive adhesive, but other adhesives known to the art can be used, including but not limited to, natural, isobutyl and butyl rubber compositions and acrylate-based adhesives and pressure sensitive adhesives. The configuration of the patch for the sustained delivery of active compound(s) to the skin can be adhesive matrix, liquid or solid state reservoir or polymer matrix. In an adhesive matrix type patch, there is an impermeable backing, a matrix comprising the active compound(s), optionally comprising a permeation enhancer and/or an anti-irritant, and a release liner.

In most transdermal patches, thin, flexible occlusive films serve as protective backing substrate and release liner. For the present application, an occlusive protective backing substrate is preferred over a non-occlusive backing substrate. The materials used for liner and backing provide storage stability by keeping the active compound(s) from migrating into or through the backing material and liner before use. The patches of the present invention desirably have the following tape properties: release or peel force <50 g/cm; tack value >50 g/cm; adhesion force 100-1200 g/cm; release force <1 g/cm; preferably the adhesion force is about 50-300 g/cm and the shear force is about 14 kg/6.25 cm². Preferably, the adhesive is medical grade. The peel force required to remove the release liner from the patch should be sufficient to prevent inadvertent separation of the liner from the patch before use and low enough so that it can be readily removed by the intended user.

Where an acrylic adhesive is used, that adhesive is medical grade and rated between 0 and 2, preferably between 0 and 1 on the Draize Code Scale. The acrylic adhesive can optionally include a cross-linking agent.

Liquid and solid state reservoir transdermal delivery devices are configured so that the reservoir comprising the active compounds, enhancers and any other included formulation compounds are located between the backing material and the adhesive, and during use, formulation compounds pass through the adhesive and then into the skin. Compatibility of various excipients and penetration enhancers with adhesives are well known to the art, and the skilled artisan can readily choose suitable concentrations and combinations of compounds and adhesives.

A typical non-silicone polymer matrix transdermal delivery device has a rim of adhesive so that the penetration enhancer, active compound(s) and other formulation compounds are not fully in contact with the adhesive. In the preferred embodiment, the entire patch is adhesive and contains at least one active compound. One surface is applied to the intended portion of the skin with gentle pressure to promote adhesion, after removal of a release liner. The other surface (away from the skin) is covered with a protective backing during storage before use and during use.

Where desired, the skin care patches of the present invention optionally comprise formulation compounds which either increase or decrease the release rates and/or absorption rates of the active compounds. Water soluble additives which increase release rate include ethylene glycol, glycerin, polyethylene glycols 200, 400, 600; polysorbate 80, lactose, gelatin, sucrose, sodium alginate, carboxymethyl cellulose, ammonium chloride, and polyvinylpyrrolidone. Lipid soluble additives which tend to increase release rate include cholesterol. Certain surfactants also have the effect of increasing release rate; these surfactants include sodium lauryl sulfate, dodecyltrimethylammonium chloride and azone. Release rates can be decreased by the addition of compounds including such fillers as kaolin, Sephadex G-25 (high pressure liquid chromatography gel filtration resin) and silica.

A preferred embodiment utilizes ascorbic acid and sodium ascorbate in a dry powder form suspended in a dry matrix. The ascorbic acid and sodium ascorbate in such a form is highly concentrated the 85-88% range, more so than in common liquid compositions, and is buffered to a pH of about 5.5 with the dry powder sodium ascorbate. The system thus is capable of applying highly concentrated ascorbic acid but within a skin-tolerable pH. The natural moisture of the skin causes the powdered compound to dissolve from the dry matrix and be absorbed at the skin surface. The occlusive protective backing ensures that the active compound(s) is (are) isolated from the atmosphere during the treatment procedure. Similar systems could employ other antioxidants such as potassium-, calcium- or magnesium-ascorbate or and ascorbate palmitate.

Preferably, the transdermal or intradermal delivery devices (patches) are shaped specifically for the target skin area to be treated.

The patch itself is preferably made of polymeric material which is chemically and biologically inert, non-toxic, non-irritating, non-sensitizing, non-allergenic and has adhesive properties which are easily manipulated. The patch material should be flexible, with good cohesive strength (shear strength of >5 kg/6.25 cm), suitable and easily controlled tack properties, low release force so that it can be readily removed from the liner backing and easily manipulated skin adhesion. The patch should have tack and adhesive properties which allow rapid adherence to the skin after minimal application of gentle hand pressure, and the matrix should rapidly mold itself closely with the contours of the target skin for best transfer of active compound(s). Adhesive properties can be determined using techniques well known to the art, for example using a digital probe tack tester (e.g., Polyken®, Testing Machines, Amityville, N.Y.) and as described, for example, in Pfister et al., Pharmaceutical Technology, January 1992, pp. 42-46. The desired adhesion of the pressure sensitive adhesive is between about 50 and 300 g/cm, preferably 80-300 g/cm. Greater adhesion is too aggressive to the skin. If the adhesion is below this level, the patch may fall off. An impermeable film is preferably bound to the surface of the patch destined to be away from the skin during use; a release liner is preferably bound to the surface of the patch destined to be applied to the skin during use, and the release liner is removed prior to use. The impermeable film is not permeable to the active compound(s), but it may be occlusive, or more preferably, nonocclusive. It is within the skill in the art to manipulate the adhesive composition in combination with the active compound(s) so as to maintain desirable adhesive properties and effective delivery of the active compound(s).

### OTHER APPLICATIONS OF THE INVENTION

The compounds of the present invention can be tested in guinea pigs which exhibit a similar melanocyte distribution more like humans (than do mice or other rodents). Guinea pig melanocytes are not just confined to hair follicles like in mice or rats but instead are also interspersed throughout the epidermis (Dover J S, et al., 1989, Arch Dermatol 125:43-9). See also, the Gilchrest et al. patents, supra.

The compounds may further be applied alone or in combination with other sunless tanning formulations, for example, commercially available bronzers, or melanogenesis stimulating agents like those disclosed in the Gilchrest et al. patents (supra) or the Fuller et al., patent (supra), all of which are incorporated by reference in their entirety.

### EXAMPLE I

### Materials and Methods

Cell Lines, Reagents and Treatments

Human melanoma cell lines were obtained from NCI-ADS and cultured as described in Monks, A et al., 1991, J. Natl. Canc. Inst. 83:757-766. Normal neonatal human epidermal melanocytes (NHEM 2489) were purchased from Clonetics and cultured in Melanocyte Growth Medium-3 (Clonetics) as suggested by the manufacturer. Purified recombinant protective antigen, B. anthracis lethal factor (LF) and B. anthracis edema factor (EF) were used to treat cells at 0.1 μg/ml each in the appropriate culture medium. Control groups were treated with PA alone, which functions as a translocator for LF and EF (Leppla, supra; Duesbery, supra). When treating with LF or EF, their respective “toxin” complexes that included PA were used (“LF+PA: LeTx” and “EF+PA: EdTx”; see FIG. 2).

PD98059 (New England BioLabs or Cell Signaling Technology, Inc.) was dissolved in dimethyl sulfoxide (DMSO), and 20 mM aliquots were stored at −20° C. To maintain sustained inhibition of MEK, PD98059 was directly added to the cultures every 24 hours to achieve a final concentration of 20 μM for the duration of the study, unless otherwise indicated. IBMX (Calbiochem Co.) dissolved in DMSO was added to the appropriate culture medium at a final concentration of 200 μM. An equivalent volume of DMSO solvent was added to controls.

Determination of Melanin Content

Intracellular melanin was solubilized by lysing cells in 0.2N NaOH and incubating at 60° C. for 1 hour. The relative absorbance at 405 nm for melanin to that at 280 nm for protein was calculated (A₄₀₅/A₂₈₀). The control relative absorbance, “A₄₀₅/A₂₈₀ (Protective antigen, “PA” alone)” was set to 1.0. The melanin content in each treatment group was expressed as the ratio:

$$\frac{{A}_{405} / {A}_{280} ( experimental )}{{A}_{405} / {A}_{280} ( PA alone )}$$

To visualize melanization, 4×10⁶ cells from each group were concentrated in a well of 96-well microplate and photographed.

### EXAMPLE II

### Induction of Melanogenesis in Human Epidermal Melanocytes

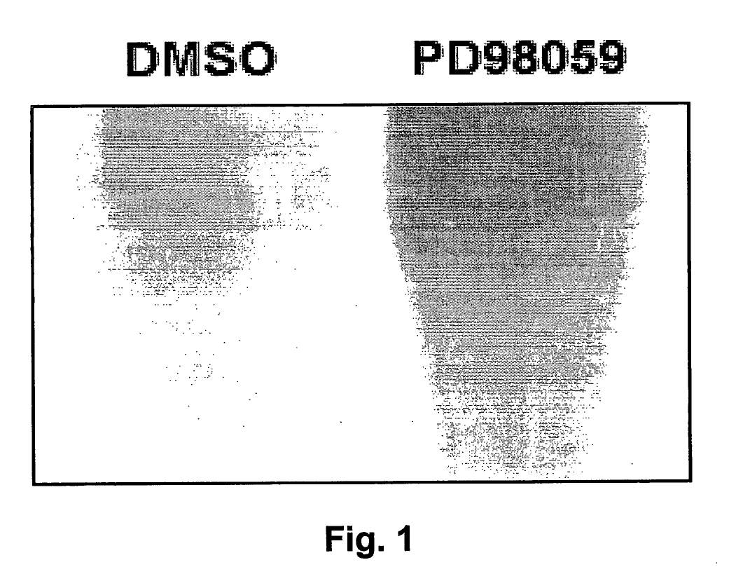

PD98059, prepared and administered for 72 hours as in Example I, induced significant melanin production in human epidermal melanocytes versus a DMSO control (FIG. 1). Inspection of the FIG. 1 clearly shows the darker color of the lysed cell solution of treated group as compared to the control group. Melanogenesis content of a cell lysate or (an intact pellet) can be adjudged visually or by spectrophotometry.

### EXAMPLE III

### Induction of Melanogenesis in Human Epidermal Melanoma Cells

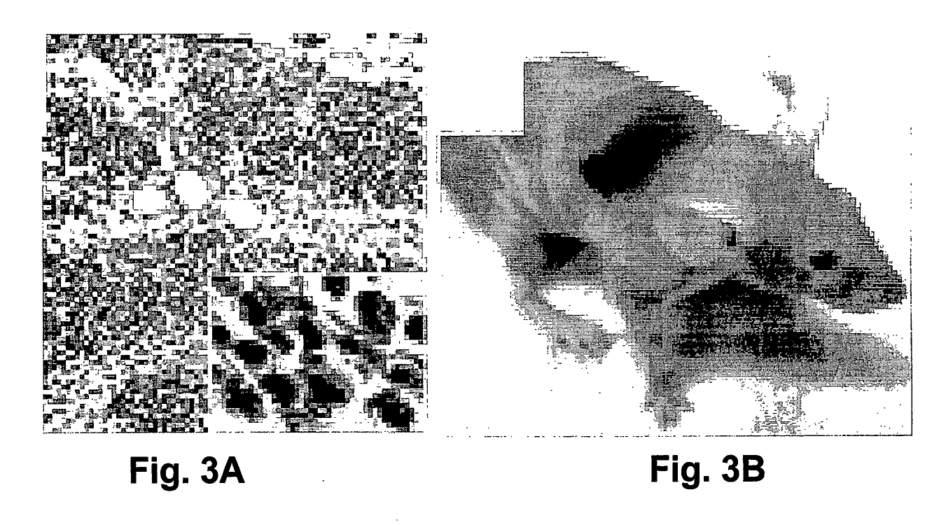

UACC-257 (FIG. 2A) and MALME-3M (FIG. 2B) melanoma cells were treated with B. anthracis lethal toxin (B. anthracis lethal factor together with protective antigen); a MEK-directed protease, or with PD98059. A dramatic increase in melanin production was detected 72 hours after treatment—cells and medium turned dark brown (FIG. 2A-2B). Consistent with these findings, human melanoma cells grown in athymic nude mice treated with B. anthracis lethal toxin, showed the formation of melanin deposits in the tumor tissue grown in surrounding murine tissues (FIG. 3A-3B).

While cAMP-elevating agents B. anthracis edema toxin (EdTx: edema factor+protective antigen); an adenylyl cyclase, and the phosphodiesterase inhibitor IBMX, alone did not stimulate de novo melanin production by human melanoma cells, each of these two agents showed synergistic effects when used together with the MEK-inhibitors B. anthracis LeTx or PD98059 and significantly enhanced melanogenesis (FIG. 2A-2B).

Cutaneous B. anthracis is characterized by the formation of a blackened eschar in the center of skin lesions, from which B. anthracis derives its name (the Greek Anthrakos means “coal”). FIG. 3A-3B show that the blackened coloration in treated melanoma cells was the result of increased melanogenesis in melanoma cells stimulated by the MEK-inhibitor Bacillus anthracis lethal toxin and further enhanced by the cAMP-elevating agent Bacillus anthracis edema toxin.

The inhibition of MAPK signaling by a MEK-inhibitor and the consequential induction of melanogenesis in normal human epidermal melanocytes as well as in human melanoma cells indicates that the MAPK pathway is a negative regulator of melanin production in human melanocytes. The present inventors predicted that exposure to sunlight (UV irradiation) stimulates melanin production in human skin melanocytes by inhibiting MAPK activity together with elevating cAMP levels, consequently leading to a tanned appearance. The inventors further suggested that the formation of “liver spots” and other hyperpigmented areas of skin in aging humans may reflect the age-related loss of MEK/MAPK activity which normally holds melanogenesis in check, at least in Caucasian skin. In a similar vein, freckles may result from a genetic loss of MBK/MAPK activity, e.g., due to a change in MEK1/2, that results in mosaicism so that localized proliferation of melanocytes in which such a loss has occurred result in the appearance of freckles. This is more likely to occur in an individual who is heterozygous for a gene or genes responsible for MAPK expression. Exposure to sunlight, i.e., UV irradiation, may induce loss of MAPK activity in the induced cells as well as serving as a stimulus for proliferation, resulting in multiplication of freckles, or enlargement and “fusion” of multiple freckles.

### EXAMPLE IV

### A Cosmetically Acceptable Emulsion of MEK Inhibitor

Three different phases are combined to form the preparation (emulsion) as follows:

| Phase A: | MEK protease/inhibitor | 1.0 μg-1.0 | g |
|---|---|---|---|
| | (optional cAMP-elevating agent) | 1.0 μg-1.0 | g |
| | Distilled water | 10.0 | g |
| Phase B | 1-oleoyl-2-acetyl-glycerol | 0.5 | g |
| | Miglyol 812 | 10.0 | g |
| Phase C | Excipient for a final mass of | 100.00 | g* |

*(including Miglyol 812 10% and Carbopol 1342 2%)

Phase A is prepared by mixing the components at room temperature. For Phase B, a moderate heating at 30° C. is preferred. Phase A and Phase B are emulsified into Phase C by standard emulsifying processes, known in the art. The resulting emulsion, when applied to the skin, will stimulate tanning even without specific sunlight exposure.

### EXAMPLE V

### A Liposomal Gel of a MEK Inhibitor

Two different phases are combined to form the preparation.

| Phase A | MEK-protease or small molecule inhibitor | 1.0 ng-1.0 | g |
|---|---|---|---|
| | (optional cAMP-elevating agent) | 1.0 ng-1.0 | g |
| | Soya phospholipids | 0.9 | g |
| | β-sitosterol | 0.1 | g |
| | Water sufficient for a final mass of | 50.0 | g |

| | |
|---|---|
| Phase B: | Gel sufficient for final mass of 50 g (Carbopol 941 gel |
| | at 2% in water) |

In a first step, a spray-dried powder is prepared from a solution of phospholipids and β-sitosterol (Phase A) according to a process such as that described in U.S. Pat. No. 4,508,703, herein incorporated by reference. This powder is then dispersed into an aqueous solution of the other ingredients in Phase A, stirred for one hour, and homogenized under pressure, according to a process such as that described in U.S. Pat. No. 4,621,023 (also incorporated by reference) to obtain a liposomal suspension. The liposomal suspension is then mixed with the same weight of Carbopol gel (Phase B).

The resulting liposomal gel can be applied, preferably once a day in the morning, on the skin to obtain a cosmetic tanning response or, in the case of desired sun exposure, to protect against pain or other symptoms resulting from that exposure.

Dosage and duration requirements may vary between subjects due to body type, initial level of pigmentation, or desired final pigmentation. Therefore, frequency and/or exact dosage may be adjusted appropriately.

The references cited above are all incorporated herein by reference, whether specifically incorporated or not.

Having now fully described the invention, it will be appreciated by those skilled in the art that the same can be performed within a wide range of equivalent parameters, concentrations, and conditions without departing from the spirit and scope of the invention and without undue experimentation.

1. A method of increasing pigmentation in human skin comprising administering to human skin a melanogenesis-stimulating effective amount of an inhibitor of the expression or the activity of an enzyme of the MAPK cellular signaling pathway in epidermal melanocytes, wherein inhibition of the expression or activity results in greater melanogenesis in the melanocytes, thereby increasing the pigmentation.

2. The method of claim 1 wherein said MAPK pathway inhibitor is a MEK-inhibitor that inhibits MEK expression or activity in epidermal melanocytes, wherein inhibition of MEK activity results in greater melanogenesis in the melanocytes, thereby increasing the pigmentation.

3. The method of claim 2, wherein the MEK activity inhibitor is selected from the group consisting of:
(a) PD98059;
(b) U0126;
(c) PD184352;
(d) an active analogue or derivative of any of (a)-(c).

4. The method of claim 2, wherein the inhibitor or MEK expression or activity is a MEK-directed protease.

5. The method of claim 4, wherein the MEK-directed protease is Bacillus anthracis lethal factor administered as Bacillus anthracis lethal toxin.

6. The method of any of claims 1-5, further comprising administering to the human skin, before, together with, or after the MAPK pathway inhibitor, a melanogenesis-stimulating or promoting amount of a cAMP-elevating agent that increases the level of intracellular cAMP.

7. The method of claim 0, wherein the MAPK pathway inhibitor and the cAMP elevating agent are administered in a single, combined formulation.

8. The method of claim 7 wherein the MAPK pathway inhibitor is a MEK inhibitor.

9. The method of claim 6 wherein the cAMP elevating agent is an adenylyl cyclase, a stimulator of adenylyl cyclase activity, a adenosine diphosphate ribosyltransferase, or a phosphodiesterase inhibitor.

10. The method of claim 9 wherein the adenylyl cyclase is Bacillus anthracis edema factor.

11. The method of claim 9 wherein the phosphodiesterase inhibitor is isobutylmethylxanthine (IBMX).

12. The method of claim 9 wherein the adenylyl cyclase stimulator is forskolin.

13. The method of claim 9 wherein the adenosine diphosphate ribosyltransferase is cholera toxin.

14. The method of any of claims 1-5, wherein the administering is topical, intradermal or transdermal.

15. The method of claim 6, wherein the administering is topical, intradermal or transdermal.

16. The method of claim 7, wherein the administering is topical, intradermal or transdermal.

17. The method of claim 8, wherein the administering is topical, intradermal or transdermal.

18. The method of claim 9, wherein the administering is topical, intradermal or transdermal.

19. A method of increasing pigmentation in human skin comprising administering to human skin a pigmentation-increasing effective amount of a combination of
(a) a MAPK pathway inhibitor and
(b) a cAMP-elevating agent,
wherein (i) inhibition of expression or activity of an enzyme of the MAPK pathway and (ii) elevation of intracellular cAMP in epidermal melanocytes stimulate melanogenesis, thereby increasing the pigmentation.

20. The method of claim 19 wherein the MAPK pathway inhibitor is a MEK inhibitor that inhibits expression or activity of the MEK enzyme.

21. The method of claim 20, wherein the inhibitor of MEK activity is selected from the group consisting of:
(a) PD98059;
(b) U0126;
(c) PD184352;
(d) an active analogue or derivative of any of (a)-(c).

22. The method of claim 20, wherein the inhibitor of MEK expression or activity is a MEK-directed protease.

23. The method of claim 20, wherein the MEK inhibitory protease is Bacillus anthracis lethal factor administered as Bacillus anthracis lethal toxin.

24. The method of any of claims 19-23, wherein the cAMP elevating agent is an adenylyl cyclase, a stimulator of adenylyl cyclase activity, an adenosine diphosphate ribosyltransferase, or a phosphodiesterase inhibitor.

25. The method of claim 24, wherein the adenylyl cyclase is Bacillus anthracis edema factor administered as Bacillus anthracis edema toxin.

26. The method of claim 24, wherein the phosphodiesterase inhibitor is isobutylmethylxanthine.

27. The method of claim 24, wherein the adenylyl cyclase stimulator is forskolin.

28. The method of claim 24, wherein the adenosine diphosphate ribosyltransferase is cholera toxin.

29. The method of any of claims 19-23, wherein the administering is topical, intradermal or transdermal.

30. The method of claim 24, wherein the administering is topical, intradermal or transdermal.

31. A pharmaceutical composition formulated for topical application to human skin for enhancing skin pigmentation comprising:
(a) an effective amount of a MAPK pathway inhibitor;
(b) optionally, a cAMP elevating agent; and
(c) a cosmetically and/or pharmaceutically acceptable carrier suitable for topical administration.

32. The pharmaceutical composition of claim 31 wherein the MAPK pathway inhibitor is a MEK inhibitor.

33. The pharmaceutical composition of claim 31 wherein the MAPK pathway inhibitor is a Raf inhibitor.

34. The pharmaceutical composition of claim 31 wherein the MAPK pathway inhibitor is an ERK1/2 inhibitor.

35. The pharmaceutical composition of claim 32, wherein the MEK-inhibitor is selected from the group consisting of:
(a) PD98059;
(b) U0126;
(c) PD184352;
(d) an active analogue or derivative of any of (a)-(c).

36. The pharmaceutical composition of claim 32, wherein the MEK-inhibitor is a MEK-directed protease.

37. The pharmaceutical composition of claim 36, wherein the protease is Bacillus anthracis lethal factor administered as lethal toxin.

38. A pharmaceutical composition formulated for intradermal or transdermal administration to human skin for enhancing skin pigmentation comprising:
(a) an effective amount of a MAPK pathway inhibitor;
(b) optionally, a cAMP elevating agent; and
(c) a pharmaceutically or cosmetically acceptable carrier suitable for intradermal or transdermal administration.

39. The pharmaceutical composition of claim 38 wherein the MAPK pathway inhibitor is a MEK inhibitor.

40. The pharmaceutical composition of claim 38 wherein the MAPK pathway inhibitor is a Raf inhibitor.

41. The pharmaceutical composition of claim 38 wherein the MAPK pathway inhibitor is an ERK1/2 inhibitor.

42. The pharmaceutical composition of claim 39, wherein the MEK-inhibitor is selected from the group consisting of:
(a) PD98059;
(b) U0126;
(c) PD184352;
(d) an active analogue or derivative of any of (a)-(c).

43. The pharmaceutical composition of claim 39, wherein the MEK-inhibitor is a MEK-directed protease.

44. The pharmaceutical composition of claim 43, wherein the protease is Bacillus anthracis lethal factor administered as lethal toxin.

45. The pharmaceutical composition of any of claims 31-44 that includes the cAMP-elevating agent, which agent is an adenylyl cyclase, an adenylyl cyclase stimulator, a phosphodiesterase inhibitor.

46. The pharmaceutical composition of claim 45, wherein the adenylyl cyclase is B. anthracis edema factor.

47. The pharmaceutical composition of claim 45, wherein the phosphodiesterase inhibitor is isobutylmethylxanthine.

48. The pharmaceutical composition of claim 45, wherein the adenylyl cyclase stimulator is forskolin.

49. A method of sunless tanning comprising administering topically to human skin a pigmentation enhancing effective amount of the pharmaceutical composition of any of claims 31-37.

50. A method of sunless tanning comprising administering to human skin a pigmentation enhancing effective amount of the topical pharmaceutical composition of claim 45.

51. A method of sunless tanning comprising administering intradermally or transdermally to human skin a pigmentation enhancing effective amount of the pharmaceutical composition of any of claims 38-44.

52. A method of sunless tanning comprising administering intradermally or transdermally to human skin a pigmentation enhancing effective amount of the pharmaceutical composition of claim 45 formulated for intradermal or transdermal delivery.
